(19) United States
(12) Patent Application Publication
(10) Pub. No.: US 2019/0145890 A1
(43) Pub. Date: May 16, 2019

(54) TIME-DIVISION MULTISPECTRA DETECTION DEVICE AND METHOD

(71) Applicant: SOLID YEAR CO., LTD., NEW TAIPEI CITY (TW)

(72) Inventor: Wei-Hsin TSAI, NEW TAIPEI CITY (TW)

(21) Appl. No.: 15/858,567

(22) Filed: Dec. 29, 2017

(30) Foreign Application Priority Data
Nov. 10, 2017 (TW) 106138968

(51) Int. Cl. G01N 21/31; G01J 3/28; G01J 3/10; G01N 21/359; A61B 5/00; G06K 9/62; G06K 9/00

(52) U.S. Cl. CPC G01N 21/3151; G01J 3/2823; G01J 3/10; G01N 21/359; A61B 5/0077; G06K 9/6202; A61B 2503/04; G06K 9/6232; G06K 9/628; G01J 2003/2826; G01J 2003/106; G01N 2021/3181; G06K 9/00369

(57) ABSTRACT

The invention provides a time-division multispectra detection device and method. The time-division multispectra detection device includes a plurality of light-emitting units of different wavelengths sequentially activatable by a control unit to emit one same wavelength of light onto an object at one same time. When one light-emitting unit is activated, the control unit synchronously activates the digital image sensing unit to obtain an image data of the object corresponding to one single wavelength of light from one respective light-emitting unit. After every spectrum image data is collected. A judgment of the human physiological characteristics of the object is performed, effectively reducing the overall costs.

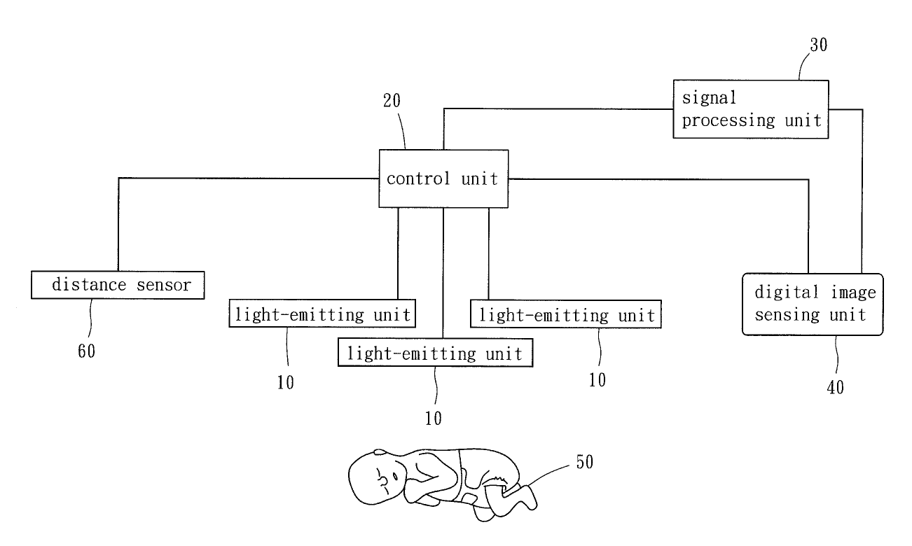

### BACKGROUND OF THE INVENTION

### 1. Field of the Invention

The present invention relates to detection technology and more particularly, to a time-division multispectra detection device and method.

### 2. Description of the Related Art

In order to improve the sleep safety of infants and young children and to prevent sudden infant death syndrome (SIDS), various kinds of monitoring devices have been created and widely used for infant care. A simple monitoring device is the use of a digital camera for recording images of infants in a long time, and the digital images thus obtained are than transmitted through the Internet to the cloud server or the parent's computer or mobile phone, so that the parent can observe any abnormal situation of the baby. There are also known physiological signal monitoring methods by directly attaching electrodes to baby body for physiological signal monitoring.

U.S. Pat. No. 7,611,472 discloses an apnea monitor that uses new designed gas flow sensor and gas differential flow sensor to detect actual airflow from patients nose and mouth to detect breathing. U.S. Pat. No. 5,864,291 and U.S. Pat. No. 5,400,012 disclose a device for monitoring the breathing of a subject that is self-contained within an enclosure and includes a strap for wrapping around the subject to thereby secure the enclosure to the subject. U.S. Pat. No. 4,146,885 discloses a hospital bed or mattress for neonatal infants with a respiration monitor and alarm to detect apnea.

The above-described various prior art monitors and devices are commonly expensive and complicated to operate. And more importantly, they fail to accurately detect the status of infants and, consequently, fail to provide early warning.

### SUMMARY OF THE INVENTION

The present invention has been accomplished under the circumstances in view. It is the main object of the present invention to provide a time-division multispectra detection device and method, which accurately detects and determines the physiological characteristics of the detected object and effectively reduces the overall cost.

To achieve this and other objects of the present invention, a time-division multispectra detection device comprises at least two light-emitting units, a control unit and a digital image sensing unit. The at least two light-emitting units are configured to emit different wavelengths of light. The control unit is electrically coupled with the at least two light-emitting units and adapted for sequentially activating the at least two light-emitting units, enabling only one light-emitting unit to emit one same wavelength of light onto an object at one same time. The digital image sensing unit is electrically coupled to the control unit. The control unit drives the digital image sensing unit to obtain an image data from the object in ambient light before activating the at least two light-emitting units, and then synchronously drives the digital image sensing unit to obtain a respective image data from the object when driving each light-emitting unit to emit light.

Preferably, the time-division multispectra detection device further comprises a image processing unit electrically coupled with the digital image sensing unit and the control unit for receiving image data from the digital image sensing unit and processing the received image data for comparison with predetermined human physiological characteristic reference information.

Preferably, the digital image sensing unit is configured to first transmit obtained image data to the image processing unit for converting to some feature space, said a normalized difference index (NDI), and then, the normal difference index is separated using a classification algorithm so that the image data after the separation generate respective comparison information according to the locations of different areas for determining whether the respective comparison information in the respective areas are identified in line with the predetermined human physiological characteristics.

Preferably, the time-division multispectra detection device further comprises a distance sensor electrically connected to the control unit, and controllable by the control unit to detect the distance between the distance sensor and the object for setting the parameters of the at least two light-emitting units and the digital image sensing unit.

To achieve this and other objects of the present invention, a time-division multispectra detection method used in the time-division multispectra detection device described above comprises the step of enabling the control unit to drive the digital image sensing unit to obtain an image data from the object in an ambient light source for use as a base data before driving the at least two light-emitting unit, the step of controlling the control unit to sequentially activate the at least two light-emitting units for enabling only one light-emitting unit to emit one same wavelength of light onto the object at one same time, the step of controlling the control unit to synchronously activate the digital image sensing unit, so that the digital image sensing unit obtains a real time spectrum image data in one-to-one correspondence between one of the at least two light-emitting units and the object, and the step of removing the base data from the real time spectrum image data for processing through an algorithm to determine whether the object have human physiological characteristics.

Other advantages and features of the present invention will be fully understood by reference to the following specification in conjunction with the accompanying drawings, in which like reference signs denote like components of structure.

### BRIEF DESCRIPTION OF THE DRAWINGS

The FIGURE illustrates system block diagram of a time-division multispectra detection device in accordance with the present invention.

### DETAILED DESCRIPTION OF THE INVENTION

First of all, it should be noted that the present invention can be widely applied to monitoring devices of various specifications and forms. Those skilled in the art can understand that the descriptive terms of the present embodiment belong to upper-level descriptions of non-limiting application such as programs, flows or electrical component terms including but not limited to descriptive contents, no limitation on the object of detection, and no limitation of detectable physiological characteristics.

The invention provides a time-division multispectra detection device, comprising a plurality of light-emitting units 10, a control unit 20, a image processing unit 30 and a digital image sensing unit 40. The light-emitting units 10 in this embodiment are made up of, for example, Near-Infrared LEDs (NIR LEDs). Each light-emitting unit 10 can consist of a single wavelength LED, or two LEDs of one same wavelength. In the present preferred embodiment, each light-emitting unit 10 consists of three LEDs that have different wavelengths. The control unit 20 is electrically coupled with the light-emitting units 10. The control unit 20 can sequentially activate each light-emitting unit 10, enabling only one light-emitting unit 10 to emit near-infrared light of a predetermined wavelength onto an object 50 at one same time. In this embodiment, the object 50 is an infant. The time-division multispectra detection device further comprises a distance sensor 60 electrically connected to the control unit 20. In this embodiment, the distance sensor 60 is a LED distance sensor. The control unit 20 controls the distance sensor 60 to detect the distance between the distance sensor 60 and the object 50, and the distance data thus detected is used for setting the parameters of each light-emitting unit 10 and the digital image sensing unit 40, enabling each light-emitting unit 10 to emit sufficient intensity near-infrared light.

The digital image sensing unit 40 is electrically connected to the control unit 20. Before activating the light-emitting units 10, the control unit 20 drives the digital image sensing unit 40 to obtain an image data from the object 50 in ambient light for use as a base image data for follow-up algorithm processing to remove background noises that are acted on the object 50 by ambient light source. When one light-emitting unit 10 is driven by the control unit 20, the control unit 20 synchronously drives the digital image sensing unit 40 to obtain a real time spectrum image data from the object 50. In this way, the captured real time spectrum image data can be individually mapped to the reflected images of the same wavelength light emitted by the light-emitting units 10 onto the object 50.

The image processing unit 30 is electrically connected to the digital image sensing unit 40. The real time spectrum image data captured by the digital image sensing unit 40 is transmitted to the image processing unit 30. After every the real time spectrum image data is collected, the image processing unit 30 first removes the base image data generated by the ambient light source to the object 50 and then converts it to a feature space, such as normalized difference index (NDI) and the like, and then, the feature space is separated using a classification algorithm such as k-means, meanshift and the like so that the image data after the separation generate respective comparison information according to the locations of different areas, so as to calculate and determine whether the respective comparison information in the respective areas are identified in line with the predetermined human physiological characteristics. The above areas can be marked as the skin area, and the above mark results can then be sent to the control unit 20 as the characteristic data.

The present invention is implemented through the above technical composition and detection method. Thus, the light-emitting units 10 can be controlled to emit different wavelengths of near-infrared light to the object 50 sequentially in a time division manner. When the light-emitting units 10 are controlled to sequentially emit different wavelengths of near-infrared light to the object 50, the object 50 reflects different image data to the digital image sensing unit 40, and the image processing unit 30 calculates and compares the respective comparison information in the respective areas to determine whether the object 50 has physiological characteristics, and to determine the area and location of the physiological characteristics. The present invention utilizes a small number of light-emitting units 10 of different wavelengths to obtain enough video data for making judgment so that the judgment result can be more accurate, enhancing the accuracy and early warning effect, and reducing the overall cost.

What is claimed is:

1. A time-division multispectra detection device, comprising:
at least two light-emitting units configured to emit different wavelengths of light;
a control unit electrically coupled with said at least two light-emitting units and adapted for sequentially activating said at least two light-emitting units, enabling only one said light-emitting unit to emit one same wavelength of light onto an object at one same time; and
a digital image sensing unit electrically coupled to said control unit;
wherein said control unit drives said digital image sensing unit to obtain an image data from said object in ambient light before activating said at least two light-emitting units, and then synchronously drives said digital image sensing unit to obtain a respective image data from said object when driving each said light-emitting unit to emit light.

2. The time-division multispectra detection device as claimed in claim 1, further comprising an image processing unit electrically coupled with said digital image sensing unit and said control unit for receiving said image data from said digital image sensing unit and processing the received said image data for comparison with predetermined human physiological characteristics.

3. The time-division multispectra detection device as claimed in claim 2, wherein said digital image sensing unit is configured to first transmit obtained image data to said image processing unit for converting to a feature space, and then, said feature space is separated using a classification algorithm so that the image data after the separation generate respective comparison information according to the locations of different areas for determining whether respective comparison information in the respective areas are identified in line with the predetermined human physiological characteristic reference information.

4. The time-division multispectra detection device as claimed in claim 1, further comprising a distance sensor electrically connected to said control unit and controllable by said control unit to detect the distance between said distance sensor and said object for setting the parameters of said at least two light-emitting units and said digital image sensing unit.

5. A time-division multispectra detection method used in the time-division multispectra detection device as claimed in claim 1, the time-division multispectra detection method comprising the steps of:
a. enabling said control unit to drive said digital image sensing unit to obtain an image data from said object in an ambient light source for use as a base data before driving said at least two light-emitting unit;
b. controlling said control unit to sequentially activate said at least two light-emitting units for enabling only one said light-emitting unit to emit one same wavelength of light onto said object at one same time;
c. controlling said control unit to synchronously activate said digital image sensing unit, so that said digital image sensing unit obtains a real time spectrum image data in one-to-one correspondence between one of said at least two light-emitting units and said object; and
d. After every the real time spectrum image data is collected, removing said base image data from said real time spectrum image data for processing through an algorithm to determine whether said object have human physiological characteristics.
